(19) United States
(12) Patent Application Publication
(10) Pub. No.: US 2017/0055897 A1
(43) Pub. Date: Mar. 2, 2017

(54) BIOFEEDBACK CHAMBER FOR FACILITATING ARTISTIC EXPRESSION

(71) Applicant: Mary-Porter Scott BROCKWAY, Charlestown, MA (US)

(72) Inventor: Mary-Porter Scott BROCKWAY, Charlestown, MA (US)

(21) Appl. No.: 15/244,645

(22) Filed: Aug. 23, 2016

Related U.S. Application Data
(60) Provisional application No. 62/210,149, filed on Aug. 26, 2015.

(51) Int. Cl. A61B 5/00; A61B 5/024; A61M 21/00; A61B 5/08; A61B 5/021; A61B 5/0478; A61B 5/01

(52) U.S. Cl. CPC A61B 5/486; A61B 5/0478; A61B 5/024; A61B 5/01; A61B 5/08; A61B 5/021; A61B 5/0077; A61B 5/0022; A61M 21/00; A61B 5/4836; A61M 2021/0027; A61M 2021/005; A61M 2021/0066

(57) ABSTRACT

Methods and systems that facilitate artistic expression using biofeedback are described. A chamber includes one or more biometric measurement devices, one or more visual displays for presenting biometric information to a user in real-time, and one or more devices for sensory stimulation, such as audio speakers, a video monitor, fans, aromatherapy devices, and heating/cooling equipment.

### CROSS-REFERENCE TO RELATED APPLICATIONS

This application claims priority to U.S. Provisional Application No. 62/210,149, filed on Aug. 26, 2015, the entire contents of which are incorporated herein by reference.

### FIELD OF THE INVENTION

This disclosure relates generally to biofeedback systems, and specifically to systems and methods that provide users with real-time biometric feedback and sensory stimuli.

### BACKGROUND

Devices for measuring and presenting biometric information are widely used for a variety of purposes. For example, electroencephalography (EEG) devices are often used for medical diagnostics and neurological research, heart-rate monitors are included in wearable fitness trackers to promote physical wellness, and many computers and smartphones incorporate fingerprint readers or retinal scanners for user authentication.

However, biometric devices have seen little use in the fields of visual, aural, and performance art. The present disclosure presents methods and systems that use such devices to facilitate artistic expression and to promote self-knowledge and introspection.

### SUMMARY

The invention provides methods and systems for providing biofeedback to a user.

Under one aspect of the invention, a method for providing biometric information to a user substantially in real-time includes measuring, using a biometric measurement device located within a chamber, biometric information pertaining to the user; transmitting the biometric information to a processing device; transforming the biometric information, using the processing device, into a human-readable representation; and displaying, on a biometric display screen in communication with the processing device, the human-readable representation of the biometric information.

Under another aspect of the invention, the biometric measurement device is an EEG device, a heart-rate monitor, a body temperature monitor, a respiration monitor, a blood-pressure monitor, or a camera.

Under another aspect of the invention, a sensory stimulus is provided to the user by a sensory stimulus device, wherein the sensory stimulus device is a an audio speaker, a video display screen, a fan, an aromatherapy device, or a heating/cooling device.

Under another aspect of the invention, the sensory stimulus device is controlled by a console that is accessible to the user.

Under another aspect of the invention, the sensory stimulus device is one of an audio speaker and a video display screen, and the console allows the user to select media from a media library to be played via the sensory stimulus device.

Under another aspect of the invention, the chamber comprises a window.

Under another aspect of the invention, the human-readable representation of the biometric information is displayed on a second biometric display screen external to the chamber.

### BRIEF DESCRIPTION OF THE DRAWINGS

For a more complete understanding of various embodiments of the present invention, reference is now made to the following descriptions taken in connection with the accompanying drawings, in which:

FIG. 1 shows the layout and contents of a biofeedback chamber, according to some embodiments of the invention.

### DETAILED DESCRIPTION

Embodiments of the present invention provide biofeedback tools that allow a user to enhance artistic expression, quantify moods and, facilitate shifts in mood. By providing real-time EEG information and other biometric data, some embodiments allow a user to identify and minimize unwanted habits, and to develop desired habits. While embodiments of the present invention may be of particular interest to professional or amateur artists, their use is not limited to any particular profession or purpose.

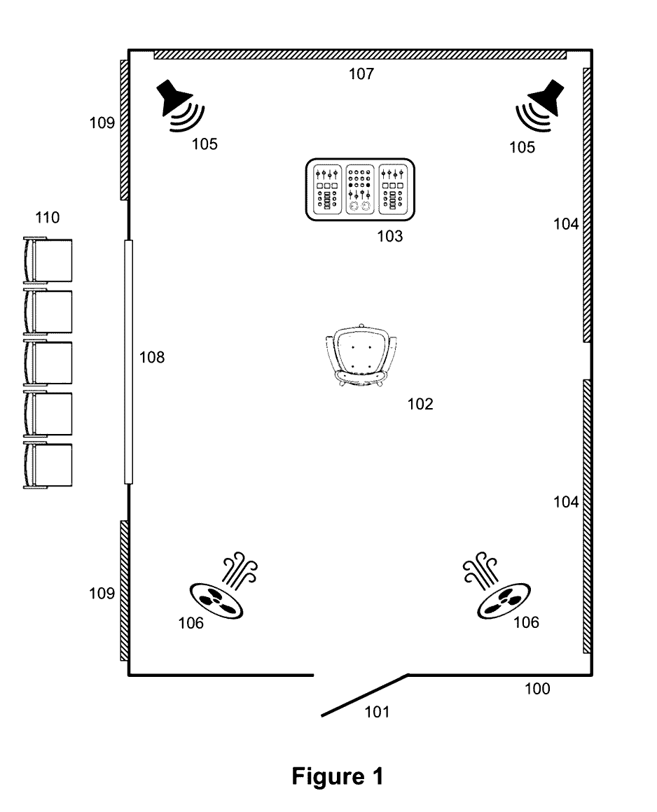

FIG. 1 is a schematic diagram showing one embodiment of the present invention. As shown in FIG. 1, chamber 100 is accessible via doorway 101. The chamber 100 must be large enough for a user to fit inside; in some preferred embodiments, chamber 100 is a rectangle 8 feet long and 10 feet wide, though the chamber may be larger or smaller as desired, and may have a variety of shapes. In some embodiments, the chamber 100 is covered by a ceiling. The chamber optionally contains a seat 102. Seat 102 may be capable of rotating (as shown in FIG. 1) allowing the user to change his or her orientation as desired.

A variety of biometric measurement devices (not shown) may be used to collect biometric information pertaining to the user. Embodiments of the present invention may make use of “electrode caps” for making EEG measurements, which allow electrodes to be placed around the user's head. Embodiments may also make use of heart-rate, body temperature, respiration, and/or blood-pressure monitors (which may be “clip-ons” or other wearable devices). Visible light and/or infrared cameras may also be used to monitor biometric signals relating to the user.

In some embodiments, the biometric information that is collected is sent to a processing device such as a computer (not shown). The biometric measurement devices may be directly connected to the processing device (via, e.g., one or more electrical wires). Alternatively, the biometric devices may transmit information to the processing device wirelessly (via, e.g., Bluetooth or WiFi), allowing the user to move around chamber 100 more freely.

The processing device receives the biometric information and transforms it into a human-readable representation, which may include graphics, text, or a combination thereof. This human-readable representation of the biometric information is then displayed on one or more biometric display screens 104 that are visible to the user. In some preferred embodiments of the invention, the biometric information is collected, transformed, and displayed in real-time, providing up-to-date biofeedback to the user. In some embodiments, the processing device may allow the biometric data corresponding to a user session to be saved using internal storage, or shared with a network-connected device (such as, e.g., a smart phone, tablet, or laptop) over network such as the Internet. In some embodiments, biometric data may be streamed over the network to viewer devices substantially in real-time.

Some embodiments further include sensory stimulus devices that provide configurable sensory stimuli to the user. As shown in FIG. 1, these sensory stimulus devices may include audio speakers 105, fans 106, and a video display screen 107. Other sensory stimulus devices that may be used include aromatherapy devices for aerial diffusion of one or more aromas, heating and cooling systems for changing the ambient temperature of chamber 100, and other devices capable of stimulating any of the senses (e.g. a device capable of inducing a specific taste).

In some preferred embodiments, the sensory stimulus devices may be controlled using a central console 103. Using the central console, a user may, e.g., select audio and/or video clips from a provided media library to be played on speakers 105 and/or display screen 107, adjust the speed and orientation of fans 106, and control any additional sensory stimulus devices that are available (e.g., adjust the ambient temperature, select a desired aroma, etc.).

As shown in FIG. 1, chamber 100 may optionally include a window 108 (made out of glass, Lucite, or any other transparent or semitransparent material) through which external viewers can see inside the chamber. The user may optionally use window 108 to draw, paint, write, or otherwise express themselves. External biometric display screens 109 may also be provided, allowing external viewers to see the same biometric information that is displayed to the user on internal biometric display screens 104. In some embodiments, audience seating 110 may optionally be provided. In some embodiments, external blinds, or another type of opaque panel, may be used to cover the window to provide privacy for the user. In some embodiments, the chamber may further contain a visible-light camera for photographing the art on the window. In some embodiments, a digital tablet on which the user may draw may be provided instead of, or in addition to, the window.

Although preferred embodiments of the present invention have been described above and shown in the accompanying figures, it should be understood that the present invention is not limited to the embodiments disclosed, but is capable of numerous rearrangements, modifications and substitutions without departing from the spirit of the invention as set forth and defined by the following claims.

What is claimed is:

1. A biofeedback apparatus comprising:
a chamber;
a biometric measurement device, located within the chamber, that is operable to measure biometric information pertaining to a user;
a processing device operable to receive biometric information from the biometric measurement device and to transform the biometric information into a human-readable representation; and
a biometric display screen, in communication with the processing device, that is operable to display the human-readable representation of the biometric information,
wherein the biofeedback apparatus is capable of measuring, transforming, and displaying biometric information to the user substantially in real-time.

2. The biofeedback apparatus of claim 1, wherein the biometric measurement device is an EEG device, a heart-rate monitor, a body temperature monitor, a respiration monitor, a blood-pressure monitor, or a camera.

3. The biofeedback apparatus of claim 1, further comprising a sensory stimulus device, wherein the sensory stimulus device is an audio speaker, a video display screen, a fan, an aromatherapy device, or a heating/cooling device.

4. The biofeedback apparatus of claim 3, further comprising a console that is accessible to the user for controlling the sensory stimulus device.

5. The biofeedback apparatus of claim 4, wherein the sensory stimulus device is one of an audio speaker and a video display screen, and wherein the console allows the user to select media from a media library to be played via the sensory stimulus device.

6. The biofeedback apparatus of claim 1, wherein the chamber comprises a window.

7. The biofeedback apparatus of claim 6, further comprising a second biometric display screen, external to the chamber, that is operable to display the human-readable representation of the biometric information.

8. A method for providing biometric information to a user substantially in real-time, the method comprising:
measuring, using a biometric measurement device located within a chamber, biometric information pertaining to the user;
transmitting the biometric information to a processing device;
transforming the biometric information, using the processing device, into a human-readable representation; and
displaying, on a biometric display screen in communication with the processing device, the human-readable representation of the biometric information.

9. The method of claim 8, wherein the biometric measurement device is an EEG device, a heart-rate monitor, a body temperature monitor, a respiration monitor, a blood-pressure monitor, or a camera.

10. The method of claim 8, further comprising: providing, using a sensory stimulus device, a sensory stimulus to the user.

11. The method of claim 9, wherein the sensory stimulus device is an audio speaker, a video display screen, a fan, an aromatherapy device, or a heating/cooling device.

12. The method of claim 10, further comprising: controlling the sensory stimulus device using a console that is accessible to the user.

13. The method of claim 12, wherein the sensory stimulus device is one of an audio speaker and a video display screen, and wherein the console allows the user to select media from a media library to be played via the sensory stimulus device.

14. The method of claim 8, wherein the chamber comprises a window.

15. The method of claim 14, further comprising displaying, on a second biometric display screen external to the chamber, the human-readable representation of the biometric information.
